(19) United States
(12) Patent Application Publication
(10) Pub. No.: US 2014/0155802 A1
(43) Pub. Date: Jun. 5, 2014

(54) CATHETER

(71) Applicant: David G. Quinn, Grayslake, IL (US)

(72) Inventor: David G. Quinn, Grayslake, IL (US)

(73) Assignee: Radius International, Inc., Grayslake, IL (US)

(21) Appl. No.: 14/000,918

(22) PCT Filed: Feb. 24, 2012
(86) PCT No.: PCT/US12/26478

§ 371 Date: Dec. 18, 2013

Related U.S. Application Data
(63) Continuation-in-part of application No. 13/035,634, filed on Feb. 25, 2011, now abandoned.
(60) Provisional application No. 61/308,653, filed on Feb. 26, 2010.

(51) Int. Cl. A61J 15/00; A61M 1/30

(52) U.S. Cl. CPC A61J 15/0073; A61M 1/30
USPC 604/6.16; 604/43; 604/30

(57) ABSTRACT

A catheter comprising a dual lumen catheter tube with a molded plastic bolus formed at the distal end of the tube. The tube and bolus assembly which results has arterial and venous ports which overlap each other longitudinally but are oriented on opposite sides of the tube and bolus assembly.

### RELATED APPLICATIONS

This PCT application is based on regular application Ser. No. 13/035,634 filed Feb. 25, 2011, and claims priority therefrom. The entire priority application is incorporated herein by reference.

### FIELD OF THE INVENTION

This invention relates in general to catheters. It relates more particularly to hemodialysis catheters. In addition, the invention relates to any corporeal catheter where aspiration and infusion are occurring simultaneously through the same multiple lumen tube.

### BACKGROUND OF THE INVENTION

Quinn U.S. Pat. No. 6,461,321 B1 and Quinn U.S. Pat. Appln. No. 2005/0182354 A1 provide background for the present application.

### SUMMARY OF THE INVENTION

The present invention introduces new concepts to hemodialysis catheters. The arterial port and the venous port are located at points where the most distal portion of the arterial port that is formed where its periphery meets the imaginary cylindrical portion of the catheter is opposite the most proximal portion of the venous port beneath it. In other words, one port partially over-laps the other. Unlike all other hemodialysis catheters, there is not any complete space between the ports. This positioning ensures that the fast and directed flow from the venous port moves quickly away from the slower and more concentrated inflow to the arterial port before they can mix, and vice versa in the reverse mode.

Both ramps serving the arterial port and the venous port are identical. In the hemodialysis catheter described in the application U.S. 2005/01182354 A1, the main portion of the arterial ramp climbed from the main longitudinal catheter axis at 21°. This angle has been determined to be ideal for directing fluid upward and forward away from the catheter body. In the new invention, the forward venous ramp now also climbs at an identical 21° rather than the previous catheter and climbed in a slightly concave arc and actually offered increased resistance to forward flow, thereby slightly increasing unwanted diffusion of flow around the bolus instead of forward an up.

In the most recent previous hemodialysis catheter, the flow to or from the arterial port was immediately directed by a sidewall or rail extending from the septum separating the two D lumens that assisted in directing the flow forward. In this new invention this same sidewall or rail is added to the distal venous lumen design to further compliment the flow directional aspects of the added 21° ramp. Both lumens now utilize the flow directional rails.

In the most recent previous catheter, the ramp of both ports incorporated a flat surface on the 21° portions of the ramps. In the new invention this flat surface, that tends to minimize flow around the sides of the ramps, has been increased and extends into the convex curve that competes the ramp areas.

To further assist in forward flow, the venous lumen has been changed from a tapering “D” to ovoid shape, to a continuous D shape in the new design. This change speeds flow and makes it more directional, thereby minimizing diffusion and resultant mixing as flow exits from the venous port.

To further accommodate the changes made to the venous port, the X and Y axis of the taper on the existing longitudinal over-molded bolus has been changed from 9° to 13°. This modification provides the ability to more easily introduce the catheter over a guide wire in a patient's vein.

The invention also relates to any corporeal catheter where aspiration and infusion are occurring simultaneously such as duel lumen gastric sumps, i.e., Salem Sumps, where gastric juices are being aspirated from the stomach in one lumen and air is being drawn into the stomach via a lumen line. If the pressure in the stomach is to remain balanced, it is vital that there is no mixing between the aspiration gastric juice lumen and the air replacement infusion lumen. The partially overlapping port design of the invention applies to any situation where infusions and aspiration are occurring with a multi-lumen catheter.

### BRIEF DESCRIPTION OF THE DRAWINGS

The invention is illustrated more or less diagrammatically in the drawings in which:

FIG. 1 is a side elevational view of a portion of a hemodialysis catheter embodying features of the invention.

FIG. 2 is a cross section view taken along line 12-12 of FIG. 11.

FIG. 3 is a top plan view of the catheter of FIG. 1.

FIG. 4 is a bottom plan view of the catheter of FIG. 1.

FIG. 5 is a longitudinal sectional view taken through the catheter of FIG. 3.

FIG. 6 is a longitudinal sectional view of a catheter being inserted over a guide wire and over an introducer sheath.

FIG. 7 is a bottom plan view of the catheter and guide wire showing their relative orientation, as the catheter is led around a turn in a patient's vein.

FIG. 8 is a longitudinal sectional view of a catheter being inserted over a guide wire with the guide wire passing along the side of the catheter tip.

FIG. 9 is a cross sectional view taken along line 9-9 of FIG. 7.

FIG. 10 is a cross sectional view of the basic catheter 12 portion of the catheter 10 without the front over-molded bolus portion 14.

FIG. 11 is a view of the catheter as shown in FIG. 1 denoting cross-sectional views.

FIGS. 12-19 are cross sectional views taken through FIG. 11.

FIG. 20 is a cross sectional view of the venous lumen showing cross sectional area as seen in FIG. 12.

FIG. 21 is a side elevational view as seen in FIG. 1 showing the cross sectional area of the arterial port periphery.

FIG. 22 is a top and bottom plan of the catheter showing the relationship and cross sectional areas of the arterial and venous ports from a top and bottom view.

FIG. 23 is formula for calculating the total effective areas of the arterial and venous ports.

FIG. 24 is a bottom plan view that shows the normal flow pattern into the arterial port and out of the venous port.

FIG. 25 is a top plan view that shows the reversed flow pattern from the arterial port and flow into the venous port.

FIG. 26 is a side view as seen in FIG. 5 that shows the relationship of the 45° skived ports and the beginning of the ramps.

### DESCRIPTION OF THE PREFERRED EMBODIMENTS

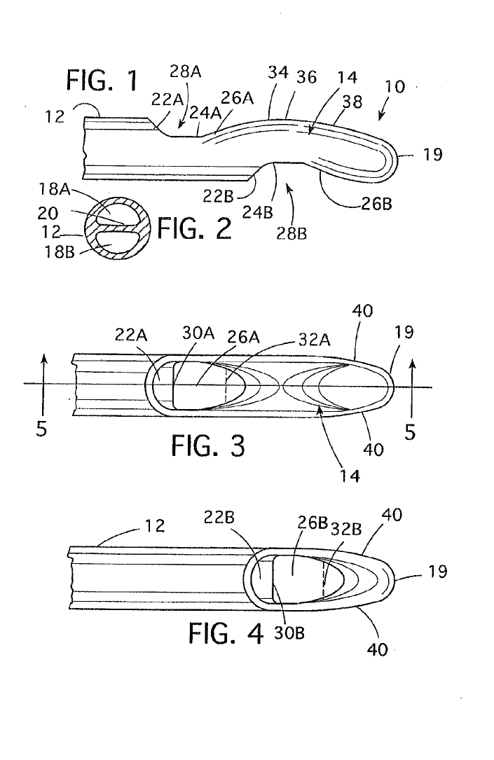

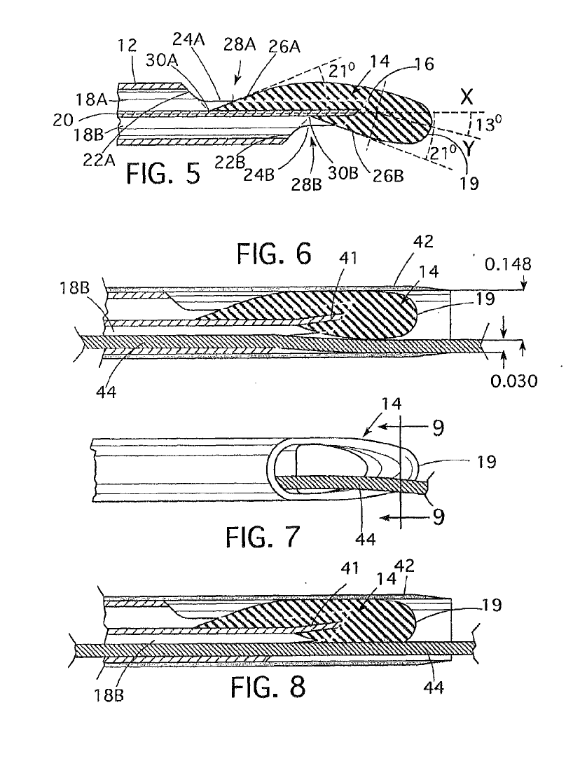

Now referring to the drawings particularly to FIGS. 1-5 and especially FIG. 5, a dual lumen catheter embodying features of the invention is illustrated generally at 10. The catheter 10 comprises a skived cylindrical extruded dual lumen tube 12 and an over-molded bolus 14 that form the complete catheter 10. The tube 12 has a distal end 16. The over-molded bolus 14 has a distal end 19.

Still referring to FIGS. 1-5, the catheter 10 is a 14.5 French tube formed of Carbothane® polyurethane material or another suitable thermoplastic polymer. The tube 12 has an outside diameter, OD, of 0.190″. The cylindrical tube 11 is divided into two identical D shaped lumens 18A and 18B by septum 20. The lumen 18A is normally referred to as an arterial lumen and the lumen 18B is referred to as a venous lumen. Each lumen 18A and 18D has a D shaped cross sectional area of about 0.0067 in².

Still referring to FIGS. 1-5, tube 12 is skived at a 45° angle to form exit port 22A for the arterial lumen 18A. The skive levels at 0.030″ from the top of septum 20 to form a wall or rail 24A that rises 0.030″ from the level of the top surface of septum 20. Likewise, the bottom side of tube 12 is skived at a 45° angle to form port 22B for the venous lumen. The venous wall or rail 24B is also skived to form a wall or rail 0.030 in height. The purpose of both the arterial rail 24A and the venous out flow rail 24B is to assist in directing the arterial inflow and venous outflow to and from the respective ports in a direct line to the port openings 22A and 22B. This flow direction is especially important in directing flow forward during the normal outflow function of the venous port 22B through the radial arterial port 28B to the ascending ramp 26B. The rails direct the flow forward and concentrate flow up and forward rather than around the edges of the over-molded bolus 14. In the reverse mode when flow is out of the arterial port 22A through the radial venous port 28A the flow is also directed forward and up over ascending ramp 26A.

Still referring to FIGS. 1-5, but more specifically to FIGS. 3, 4 and 5, ramps 26A and 26B incline at a 21° angle from the septum floor 20. The beginning of the incline of both ramps 26A and 26B is at the imaginary point 30A and 30B where the 45° angles of exit ports 22A and 22B meet the floor of septum 20. Testing has shown that the 21° angle is ideal to carry the flow in an upward direction while minimizing the spread of the fluid around the edges of the bolus tip 14, thus minimizing mixing of flow between the two lumens 18A and 18B. Both ramps have a flat straight surface to the height 32A and 32B where the surface begins to taper convexly. At this point the friction between the fluid and the bolus 14 begins to decrease as it encounters a convex rather than a flat surface 32A and 32B are at a perpendicular height of 0.049″ from the surface of septum 20.

Now referring to FIG. 1, from the top of the flat ramp 26A the bolus curves in a continuing convex arc 34 with a radius of 0.529″. Arc 34 continues to where it meets the OD of the cylinder comprising the catheter 10 at 36. Arc 34 continues past the top point 36 and meets long arc 38 that forms the long top surface of bolus 14. Arc 38 has a radius of 0.927″. The beginning 30B of ramp 26B is directly perpendicular under the top 36 of arc 34. Therefore, the beginning of venous outflow ramp 26B begins at the end of arterial radial port 28A.

Now again referring to FIGS. 5, the tip 19 of over-molded bolus 14 is has a radius of 0.066″ or an OD of 0.132″. The elongated portion of bolus 14 under the top radius 38 angles downwardly at 13° to septum 20. This angle allows the tip to present an effective bullet tip shape during insertion and in situ to prevent damage to vessel walls that can result in the build up of fibrin sheaths that could occlude the catheter 10 lumens. The distal tip 19 of the bolus 14 tapers from an OD of 0.132″ to the full OD 0.190″ of the cylindrical tube at arc 40.

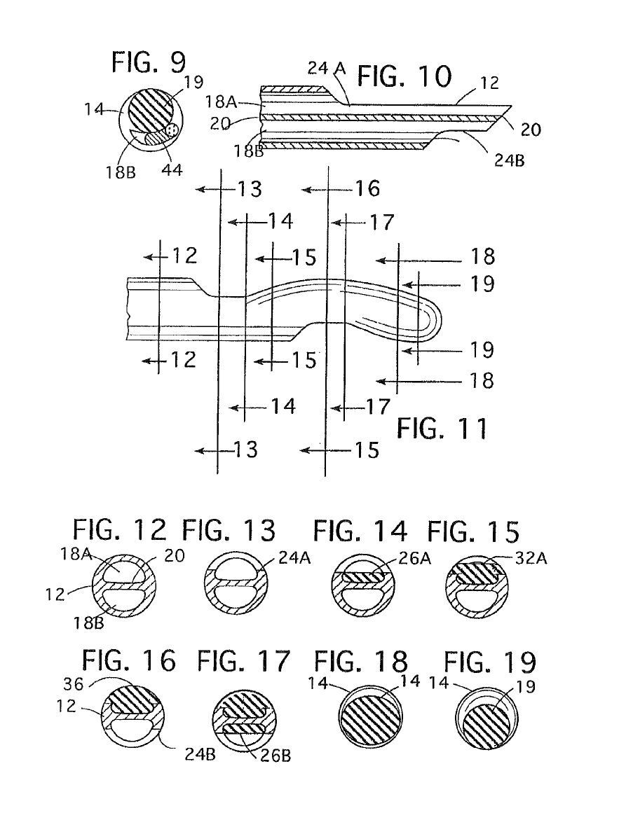

Now referring to FIGS. 5-9. The effective longitudinal axis Y of the forward portion of over-molded bolus 14 from radial point 36 to the bolus tip 19 is inclined to longitudinal axis Y at an angle of 13°. The side surfaces 40 of bolus 14 are curved inwardly to tip 19. The bolus 14 is inclined forwardly to the X axis at a angle of 77°.

The aforementioned size, shape and orientation the nose of bolus 14 provides several advantages in the use of the catheter 10. First, its smaller size facilitates easy entry into, and travel through, a patient's vein by the bolus 14. Second, the offset nose 19 section of bolus 14 places a portion of its periphery tangent to a hypothetical cylinder in which the outer surface of the bolus 14 passage lies, even though it is considerably thinner than the remainder of the bolus 14 over-molded over the tube 12. Third, when guide wire 44 insertion is employed, the nose section 19 flexes radially away from the wire where is emerges from port 22A without forcing either the nose section or the wire substantially outside of the aforementioned cylinder into the vein wall. Fourth, when traveling around curves in a vein during insertion, the bolus nose resists bending sideways and catching on the vein wall. It is also seen that the periphery of nose section 19 engages a vein wall when the top of the port 22B does. This prevents the trailing top edge of port 22B from having the vein wall wrap around it and become abraded. Likewise, The top of the arc 36 on the over-molded bolster 14 also meets the outside periphery of the imaginary catheter 10 cylinder and prevents the trailing edge of 22A from abrading the vein wall.

During insertion, as seen in FIGS. 6-9, the guide wire 44 causes the nose section to flex outwardly until its axis Y is substantially parallel to the axis X of the bolus. FIG. 6 illustrates that maximum width of the bolus 14 is 0.148″ and the guide wire has an OD of 0.0038″ for a total of 0.186″.

Now referring to FIGS. 5, 10 and 12, unlike the venous lumens described in Quinn U.S. Pat. No. 6,461,321 B1 and Quinn Application U.S. 2005/0182354 A1, the venous lumen 18B does not transition to a slightly larger oval port at port 22B. Lumen 22B maintains its same dimensions through its entire length in catheter 10. The purpose of this new configuration is to maintain flow speed and force, not to lower them. Because the invention introduces the same 21° ramp on the venous port as the arterial port it is advantageous to maintain faster flow forwardly and upwardly across flat ramp 26B. The invention also introduces a flatter and larger ramp section that transitions flow up, forward and away from the tip and therefore away from arterial inflow port 22A. In the catheters of the aforementioned Quinn patent and patent application, the leading arterial ramp also has a slightly concave curved ramp that also slows and diffuses flow. The present invention is designed to send flow forward while minimizing the slowing of flow and the diffusion of flow. Flow is meant to continue forward over the tip of the catheter tip 19.

In FIGS. 3 & 4, the flat ramps 26A and 26B raise at 21° to level 32A and 32B. At this point the center portion of the ramp remains flat to minimize the diffusion of flow around the edges of the tip. However, this flat area beyond 32A and 32B begins a concave curve to again minimize resistance to flow. This concave curve continues over the top of the catheter periphery at 36 to maintain a gradual curved surface where the periphery of the catheter 10 engages the vein wall.

In FIG. 10 is the side view of skived catheter 12. FIG. 11 is the side elevational view of catheter 10 shown in FIG. 1. which serves a vehicle for cross-sectional views of the catheter. FIGS. 12 is a cross-sectional view of the catheter at a point before it is skived. FIG. 13 shows a cross-section at the point where the top skive shows the creation of rail or wall 24A. FIG. 14 shows the beginning of ramp 26A at the point where the ramp has risen at 21° to the top of rail 24A. FIG. 15 shows the rail rising to the top of the 21° portion of ramp 26A. FIG. 16 shows the ramp at its apex at 36 and the beginning of venous ramp 26B and rail 24B. FIG.18 & 19 shows the bolus 14. The cross sectional portion of the bolus is elliptical which assists in the prevention of the tip from twisting laterally.

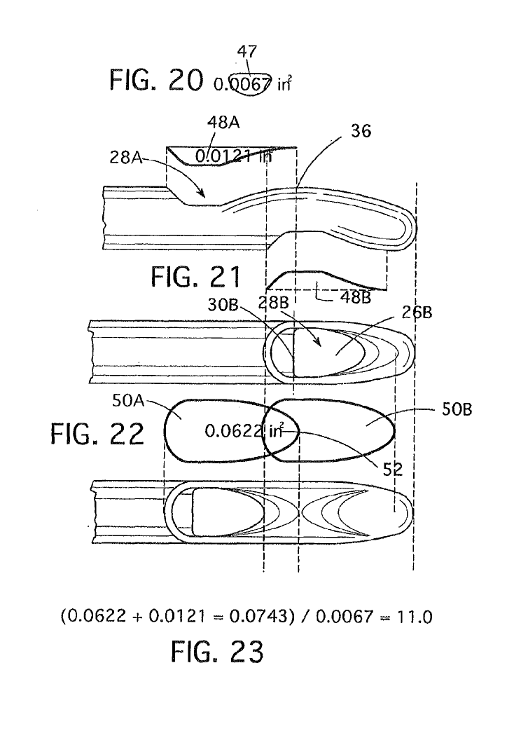

Now referring to FIGS. 20-23, the cross-sectional area 0.0067 in2 of lumen 18B is shown at 47. 48A shows the cross-sectional area of the side view of arterial port 28A within its peripheral cylindrical shape. 48B shows the identical shape and 0.0121 in2 area for 48B. The top view area and shape calculation for arterial port 48A and venous port 48B are shown as 0.0622 in2 at 50A and 50B. The ports overlap each other at 52. The beginning of ramp 26B starts at 30B. 30B is also the point of the apex of ramp 26A. Therefore the 45° skive 22B of radial port 28B begins before point 36 and port 28B begins before 28A ends. The ports are slightly superimposed over each other.

The simple formula in FIG. 23 describes that the total approximate open area for flow in both the arterial and venous ports formed by the periphery of these ports where they meet the imaginary cylinder of the catheter 10. This port area is over 10 times the cross-sectional area of either D lumen. The function of these large ports is at least fourfold. First, the size and depth of the ports prevent the vein walls from occluding the protected D lumens in the aspiration mode. Second, the tapered shape of the expanded ports eliminates any dead space for the collection of debris. Third, the shape of the ports protects the vein walls from abrasion by the trailing edges of the ports that can cause fibrin sheath build up at these edges. Fourth, in the outflow mode the blood is diffused over the bolus tip, which causes it to mix with the patient's venous blood without causing “whipping” due to concentrated flow associated with open ports. Essentially, the invention's ports have the free outflow advantages of open cut off tip ports while also protecting the port from vein damage and allowing inflow in the aspiration mode.

FIG. 26 shows the relationship between the 45° skived arterial port 22A and venous port 22B to the initiation of ramps 26A and 26B from the floor of septum 20 at points 30A and 30B. So as to not restrict flow, the ramps do not begin to rise until the septum floor exits the 45° angle of the port. The point where the flat ramp 26A meets the convex continuing arc rises 0.049″ from the floor of septum 20. The total height from the septum floor to the cylindrical OD of the catheter 10 is 0.086″. In the case of both ramp 26A and 26B this perpendicular height from the septum floor is more than 55% of the total height 0.086″. The total length of the invention from the proximal edge of port 22A to the end of tip 19 is 0.752″. The catheter tip of U.S. patent application U.S. 2005/0182354 A1 is 1.162″ in length. Moving the ports closer together results in a tip that is 35% shorter than the previous design.

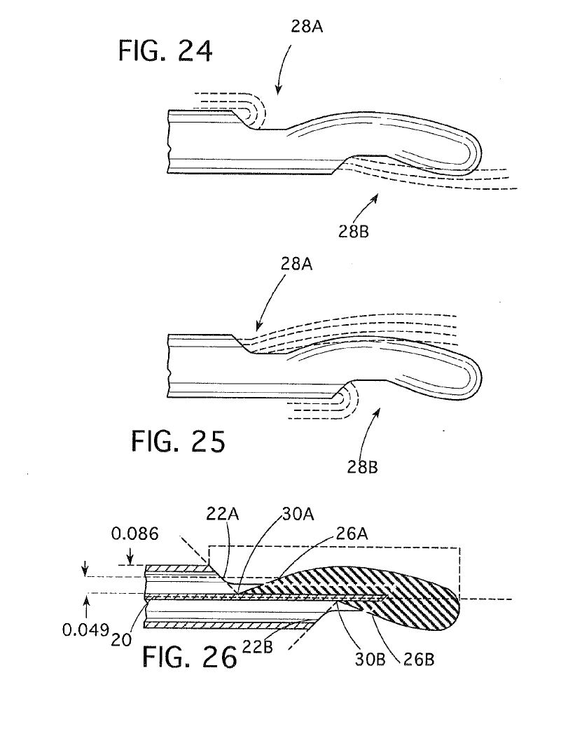

FIG. 24 shows flow in the normal use of the invention hemodialysis catheter. Blood flows out of the venous port 28B into the venous stream and returns to the lungs. The flow is forward and away from the catheter 10. Blood is aspirated into arterial port 28A and returned to the dialysis machine for cleansing before being returned to port 28B. The blood aspirated into radial port area 28A is pulled into the port from an area very proximal to the port. This fact coupled with the fact that the fluid emerging from the venous port 28B is at its highest velocity reduces the opportunity of the blood mixing between the two ports. Situations do occur where the arterial aspiration port 28A may become occluded. In this situation it is common practice to reverse the flow. This situation is shown in FIG. 25. In this invention the desirable characteristics shown in FIG. 24 still apply because the outflow ramps in both ports are identical. There are more tendencies for mixing in the reverse flow mode shown in FIG. 25 because the outflow must pass over the inflow port. In the catheter of the aforementioned Quinn patent and application, mixing in the reverse mode can be reduced from the common 40% plus rate with Mahurkar catheters to less than 7%. In this invention the mixing is reduced to near zero % in the reverse mode.

While preferred embodiments of the invention have been described, it should be understood that the invention is not so limited, and modifications may be made without departing from the invention.

1. A blood vessel catheter comprising:
a. a catheter tube containing a first lumen and a second lumen separated by a septum, said tube having a proximal and a distal end;
b. a bolus tip fastened to said tube on said distal end of said tube to form a tube and tip assembly;
c. an arterial port extending radially out of said catheter assembly in communication with said first lumen;
d. a venous port extending radially out of said catheter assembly in a direction substantially opposite to the radial direction of said arterial port and in communication with said second lumen;
e. said arterial and venous ports each being longitudinally elongated with respect to the length of said tube and tip assembly with a position of said venous port being positioned closer to said tip than said arterial port, and said venous port overlapping said arterial port along its length by at least a portion of the length of said arterial port.

2. The blood vessel catheter of claim 1 further characterized in that:
a. said arterial port includes a flow control ramp which rises up from the septum in said first lumen at an angle of substantially 21°.

3. The blood vessel catheter of claim 2 further characterized in that:
a. said venous port including a flow control ramp which rises up from the septum in said second lumen at an angle of substantially 21°.

4. The blood vessel catheter of claim 3 further characterized in that:
a. both ramps include a flat surface that extends over 55% of the total radial distance from the corresponding system surface to the outside diameter of the catheter tube.

5. The blood vessel catheter of claim 3 further characterized in that:
a. each port is bracketed by side walls approximately 0.030″ high forming rails which direct fluid flow.

6. The blood vessel catheter of claim 1 further characterized in that:
a. both the first and second lumens have a D-shape through their entire lengths.

7. The blood vessel catheter of claim 1 further characterized in that:
a. the bolus tip tapers longitudinally along its length with an inward taper angle of approximately 13° on both X and Y axes of the tip length.

8. The blood vessel catheter of claim 1 further characterized in that:
a. said catheter tube is extruded from thermoplastic polymer material; and
b. said bolus tip is over-molded from plastic on the distal end of said tube to form each of said ports.

9. An enteral feeding catheter comprising:
a. a catheter tube containing a first lumen and a second lumen separated by a septum, said tube having a proximal and a distal end;
b. a bolus tip fastened to said tube on said distal end of said tube to form a tube and tip assembly;
c. an first port extending radially out of said catheter assembly in communication with said first lumen;
d. a second port extending radially out of said catheter assembly in a direction substantially opposite to the radial direction of said first port and in communication with said second lumen;
e. said first and second ports each being longitudinally elongated with respect to the length of said tube and tip assembly with a position of said first port being positioned closer to said tip than said second port, and said first port overlapping said second port along its length by at least a portion of the length of said second port.

10. The catheter of claim 9 further characterized in that:
a. said first port includes a flow control ramp which rises up from the septum in said first lumen at an angle of substantially 21°.

11. The catheter of claim 10 further characterized in that:
a. said second port including a flow control ramp which rises up from the septum in said second lumen at an angle of substantially 21°.

12. The catheter of claim 10 further characterized in that:
a. both ramps include a flat surface that extends over 55% of the total radial distance from the corresponding system surface to the outside diameter of the catheter tube.

13. The catheter of claim 10 further characterized in that:
a. each port is bracketed by side walls approximately 0.030″ high forming rails which direct fluid flow.
